(19) United States
(12) Patent Application Publication
(10) Pub. No.: US 2025/0205477 A1
(43) Pub. Date: Jun. 26, 2025

(54) Devices, Systems, And Methods For Removing And Replacing A Subassembly Of An Electrode Assembly

(71) Applicant: NOVOCURE GMBH, Root D4 (CH)

(72) Inventors: Yoram Wasserman, Haifa (IL); Stas Obuchovsky, Haifa (IL); David Shapiro, Haifa (IL)

(21) Appl. No.: 18/989,835

(22) Filed: Dec. 20, 2024

Related U.S. Application Data
(60) Provisional application No. 63/612,729, filed on Dec. 20, 2023.

(51) Int. Cl. A61N 1/04

(52) U.S. Cl. CPC A61N 1/0492; A61N 1/0408

(57) ABSTRACT

Apparatuses for use in applying TTFields are disclosed. An apparatus includes an electrode subassembly having at least one electrode element with a skin-facing side and a skin-facing surface. The electrode subassembly has a skin-facing surface. A skin contact subassembly is removably coupled to the skin-facing surface of the electrode subassembly by a mechanical coupling. The skin contact subassembly includes an electrically conductive skin contact layer, which is configured to contact skin of a subject. When the skin contact subassembly is disposed against the skin-facing surface of the electrode subassembly, the electrically conductive skin contact layer is electrically coupled to the at least one electrode element.

### CROSS-REFERENCE TO RELATED APPLICATIONS

This application claims priority to and the benefit of the filing date of U.S. Provisional Patent Application No. 63/612,729, filed Dec. 20, 2023, the entirety of which is incorporated by reference herein.

### BACKGROUND

Tumor Treating Fields (TTFields) therapy is a proven approach for treating tumors using alternating electric fields at frequencies between 50 KHz-1 MHz, such as 100-500 kHz. The alternating electric fields are induced by electrode assemblies (e.g., arrays of capacitively coupled electrodes, also called transducer arrays) placed on opposite sides of a target location in the subject's body. When an AC voltage is applied between opposing electrode assemblies, an AC current is coupled through the electrode assemblies and into the subject's body. And higher currents are strongly correlated with higher efficacy of treatment.

The electrode assemblies used during application of TTFields typically include an electrically conductive hydrogel layer that serves as a skin contact layer that adheres to the skin of the subject. The electrically conductive hydrogel typically has a shorter lifespan than the rest of the electrode assembly. For example, with use, the skin contact layer can degrade, for example, by collecting oil and dirt, thereby reducing effectiveness of the electrically conductive hydrogel layer. The hydrogel is typically integral to the electrode assembly. Thus, upon expiration or contamination of the hydrogel, the entire electrode assembly must be disposed of and replaced.

### SUMMARY

Disclosed herein, in various aspects, are apparatuses and kits for applying TTFields.

In one aspect, an apparatus includes an electrode subassembly having at least one electrode element with a skin-facing side and a skin-facing surface. The electrode subassembly has a skin-facing surface. A skin contact subassembly is removably coupled to the skin-facing surface of the electrode subassembly by a mechanical coupling. The skin contact subassembly includes an electrically conductive skin contact layer, which is configured to contact skin of a subject. When the skin contact subassembly is disposed against the skin-facing surface of the electrode subassembly, the electrically conductive skin contact layer is electrically coupled to the at least one electrode element.

A method of using the apparatus can comprise removing the skin contact subassembly from the skin-facing surface of the electrode subassembly. The method can further comprise positioning a new skin contact subassembly against the skin-facing surface of the electrode subassembly so that the new skin contact subassembly is removably coupled to the electrode subassembly by mechanical coupling.

A kit can comprise an electrode subassembly comprising at least one electrode element having a skin-facing side and a skin-facing surface. The electrode subassembly has a skin-facing surface. The kit can further comprise a plurality of skin contact subassemblies each comprising an electrically conductive skin contact layer. The electrically conductive skin contact layer is configured to contact skin of a subject. Each skin contact subassembly of the plurality of skin contact subassemblies can be configured to be removably coupled to the electrode subassembly by mechanical coupling so that the electrically conductive skin contact layer is electrically coupled to the at least one electrode element.

A system can comprise an electrode subassembly comprising at least one electrode element having a skin-facing side and a skin-facing surface, wherein the electrode subassembly has a skin-facing surface. The system can further comprise a skin contact subassembly comprising an electrically conductive skin contact layer, wherein the electrically conductive skin contact layer is configured to contact skin of a subject. The skin contact subassembly is configured to be removably coupled to the electrode subassembly by mechanical coupling so that the electrically conductive skin contact layer is electrically coupled to the at least one electrode element.

Systems and methods for using the disclosed treatment assemblies are also disclosed.

### BRIEF DESCRIPTION OF THE DRAWINGS

FIG. 1 is a schematic top view representation of an exemplary treatment assembly as disclosed herein.

FIG. 2 is a schematic cross-sectional representation of the exemplary treatment assembly as disclosed herein, taken in the plane 2-2′ of FIG. 1.

FIG. 3 is a schematic cross-sectional representation of the exemplary treatment assembly as disclosed herein, taken in the plane 2-2′ of FIG. 1, according to another embodiment.

FIG. 4 is a schematic cross-sectional representation of the exemplary treatment assembly as disclosed herein, taken in the plane 2-2′ of FIG. 1, according to another embodiment.

FIG. 5 is a schematic cross-sectional representation of an exemplary conductive adhesive structure comprising a substrate with opposed conductive adhesive or conductive gel layers.

FIG. 6 is a schematic cross-sectional representation of an exemplary system for aligning an electrode subassembly and a skin contact subassembly of the treatment assembly of FIG. 1.

FIG. 7 is a schematic cross-sectional representation of another exemplary system for aligning an electrode subassembly and a skin contact subassembly of the treatment assembly of FIG. 1.

FIG. 8 is a schematic cross-sectional representation of another exemplary system for aligning an electrode subassembly and a skin contact subassembly of the treatment assembly of FIG. 1.

FIG. 9 is a schematic side view of hook material and loop material as disclosed herein.

FIG. 10 is a schematic side view of an exemplary reclosable fastener as disclosed herein.

Various embodiments are described in detail below with reference to the accompanying drawings, wherein like reference numerals represent like elements.

### DETAILED DESCRIPTION

This application describes exemplary electrode assemblies that can be used, e.g., for delivering TTFields to a subject's body and treating one or more cancers or tumors located in the subject's body.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

As used in the specification and the appended claims, the singular forms “a,” “an” and “the” can optionally include plural referents unless the context clearly dictates otherwise. Thus, unless the context clearly dictates otherwise, disclosure of “a layer” can represent disclosure of embodiments in which a single such layer is provided, and in other optional aspects, can represent disclosure of embodiments in which a plurality of such layers are provided.

As used herein, the term “conductive gel” should be understood to include hydrogel.

As used in the specification and the appended claims, the term “hook and/or loop material” refers to an electrically conductive hook and/or loop material. Similarly, the term “reversible reclosable fastener” refers to an electrically conductive reversible reclosable fastener.

Existing electrode assemblies for providing TTFields are unitarily constructed as an assembly with one or more electrode elements and a skin contact layer. Disclosed herein are electrode assemblies that permit replacement of the skin contact layer and subsequent reuse of the electrode subassembly.

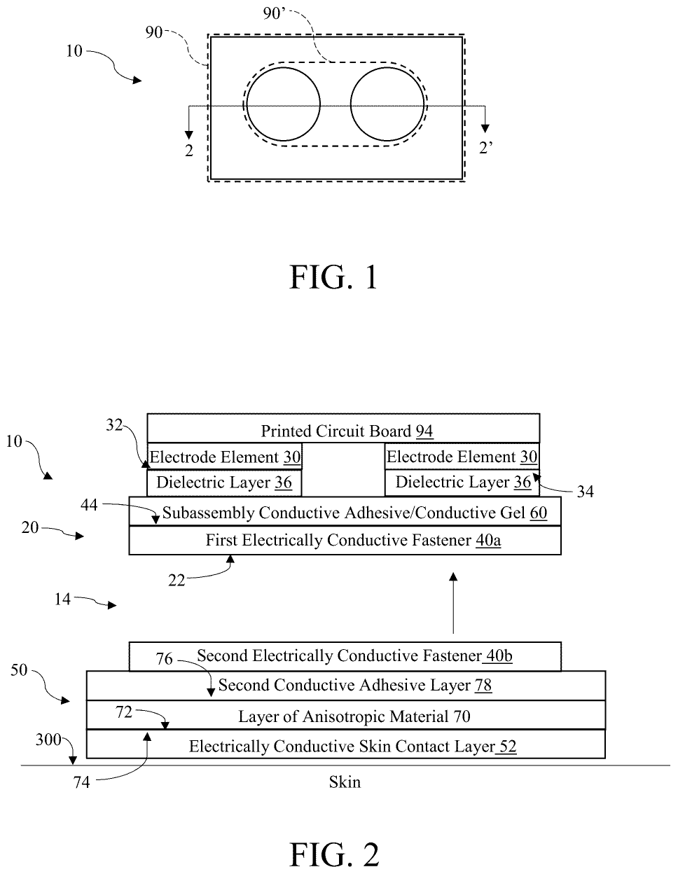

FIG. 1 illustrates a top schematic view of an apparatus 10 for providing TTFields. Referring also to FIG. 2, showing a schematic cross-sectional representation of the apparatus 10, taken in the plane 2-2′, the apparatus 10 can comprise an electrode subassembly 20 having a skin-facing surface 22. The electrode subassembly 20 can comprise at least one electrode element 30 having a skin-facing side 32 and a skin-facing surface 34.

The apparatus 10 can further comprise a skin contact subassembly 50. The skin contact subassembly 50 can be removably coupled to the skin-facing surface of the electrode subassembly by a mechanical coupling 14 (not shown, except by location). The skin contact subassembly 50 can comprise an electrically conductive skin contact layer 52. When the skin contact subassembly is disposed against the skin-facing surface 22 of the electrode subassembly 20, the electrically conductive skin contact layer 52 can be electrically coupled to the at least one electrode 30 and can be configured to contact skin 300 of a subject.

For example, the mechanical coupling 14 can be or can comprise (can be formed at least in part by) at least one electrically conductive fastener. In some exemplary aspects, the electrode subassembly 20 can comprise a first electrically conductive fastener 40a, and the skin contact subassembly 50 can comprise a second electrically conductive fastener 40b.

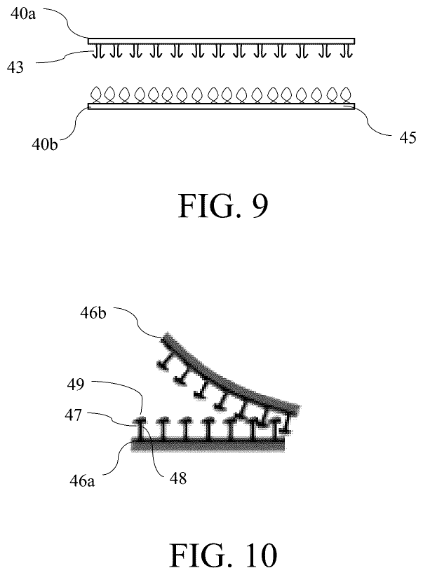

For example, in some aspects, the first electrically conductive fastener 40a can comprise (or be) one of an electrically conductive hook or loop material, and the second electrically conductive fastener 40b can comprise (or be) the other of electrically conductive hook or loop material. The one of the hook or loop material of the electrode subassembly 20 and the other of the hook or loop material of the skin contact subassembly 50 can be engaged to define the mechanical coupling. FIG. 9 shows an exemplary embodiment of the first electrically conductive fastener 40a and the second electrically conductive fastener 40b, in which the electrically conductive fastener 40a is hook material 43 and the second electrically conductive fastener 40b is loop material 45. As further described herein, in additional embodiments, the electrically conductive fastener 40a can comprise or be loop material 45 and the second electrically conductive fastener 40b can comprise or be hook material 43. An example of a hook and loop material is the VELCRO™ brand products (available from Velcro USA, Inc., Manchester, NH, USA).

In some aspects, the hook or loop material of the electrode subassembly 20 can comprise metal. In other aspects, the one of the hook or loop material of the electrode subassembly 20 can comprise a coating comprising metal. In some aspects, the one of the hook or loop material of the electrode subassembly comprises composite material having conductive particles dispersed therethrough. In other aspects the one of the hook or loop material of the electrode subassembly comprises a coating having conductive particles dispersed therethrough. The conductive particles can comprise carbon. For example, the conductive particles comprise one or more of: graphite powder, carbon flakes, carbon fibers, carbon granules, carbon nanotubes, carbon nanowires, carbon black powder, or carbon microcoils. In other aspects, the conductive particles can comprise metal.

In some aspects, the hook or loop material of the skin contact subassembly 50 can comprise metal. In other aspects, the hook or loop material of the skin contact subassembly 50 can comprise a coating comprising metal. In some aspects, the hook or loop material of the skin contact subassembly 50 can comprise composite material having conductive particles dispersed therethrough. In other aspects, the hook or loop material of the skin contact subassembly 50 can comprise a coating comprising composite material having conductive particles dispersed therethrough. The conductive particles can comprise carbon. For example, the conductive particles comprise one or more of: graphite powder, carbon flakes, carbon fibers, carbon granules, carbon nanotubes, carbon nanowires, carbon black powder, or carbon microcoils. In other aspects, the conductive particles can comprise metal.

In some aspects, and with further reference to FIG. 10, the first electrically conductive fastener 40a can comprise a first layer of an electrically conductive reversible reclosable fastener 46a, and the second electrically conductive fastener 40b can comprise a second layer of an electrically conductive reversible reclosable fastener 46b. The layer of reversible reclosable fastener can comprise a plurality of fastening elements 47 that are configured to releasably engage similar or identical fastening elements of a corresponding layer of reversible reclosable fastener. For example, the plurality of fastening elements 47 can comprise flexible projection bodies 48 and widened (e.g., mushroom-shaped) heads 49. One exemplary reversible reclosable fastener is DUAL-LOCK™ reclosable fastener provided by 3M™ of Minneapolis, MN, USA; or ALFA-LOK™ (Velcro USA, Inc., Manchester, NH, USA). The first layer of the reversible reclosable fastener of the electrode subassembly 20 and the second layer of the reversible reclosable fastener of the skin contact subassembly 50 can be engaged to define the mechanical coupling.

In some aspects, the first layer of the electrically conductive reversible reclosable fastener of the electrode subassembly and/or the corresponding second layer of the electrically conductive reversible reclosable fastener of the skin contact subassembly can comprise metal or a coating comprising metal. In some aspects, the first layer of the reversible reclosable fastener of the electrode subassembly and/or the corresponding second layer of the reversible reclosable fastener of the skin contact subassembly can comprise composite material having conductive particles dispersed therethrough or a coating having conductive particles dispersed therethrough. The conductive particles can comprise carbon. For example, the conductive particles comprise one or more of: graphite powder, carbon flakes, carbon fibers, carbon granules, carbon nanotubes, carbon nanowires, carbon black powder, or carbon microcoils. In other aspects, the conductive particles can comprise metal.

The first electrically conductive fastener 40a (e.g., the hook or loop material or reversible reclosable fastener of the electrode subassembly 20) can have an outwardly facing surface 44 that faces toward the at least one electrode element 30. In some aspects, the electrode subassembly 20 can further comprise an electrode subassembly conductive adhesive or conductive gel layer 60 disposed against the outwardly facing surface 44 of the first electrically conductive fastener 40a.

In still other exemplary aspects, the electrode subassembly 20 can comprise at least one projection, protrusion, slot, hook, fastener, snap, or interlocking feature that is configured to engage a corresponding projection, protrusion, slot, hook, fastener, snap, or interlocking feature of the skin contact assembly 50 to define the mechanical coupling 14. Generally, the mechanical coupling 14 can comprise one or more interlocking elements between the subassembly 20 and the skin contact assembly 50 that secure the electrode subassembly to the skin contact assembly.

In some optional aspects, the electrode subassembly 20 can further comprise a dielectric layer 36 on the skin-facing side 32 of the at least one electrode element 30. For example, the dielectric layer 36 can be positioned on the skin-facing surface 34 of the at least one electrode element 30. In some aspects, the dielectric layer 36 can comprise a ceramic or dielectric polymer. In aspects in which the dielectric layer 36 comprises a dielectric polymer, the dielectric polymer has a dielectric constant greater than 10 or greater than 20. In other aspects, the dielectric layer can be omitted from the electrode subassembly 20.

In some aspects, the apparatus 10 does not comprise adhesive between the skin contact subassembly and the electrode subassembly. For example, the electrode subassembly 20 and the skin contact subassembly 50 can be removably coupled by the mechanical coupling alone. In other aspects, the mechanical coupling may be supplemented by adhesive (e.g., a conductive adhesive) on one or both sides of the mechanical coupling (e.g., mechanical fastener).

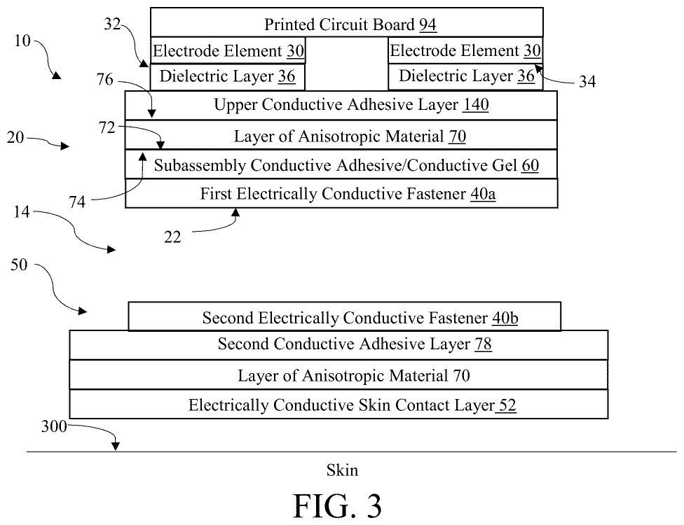

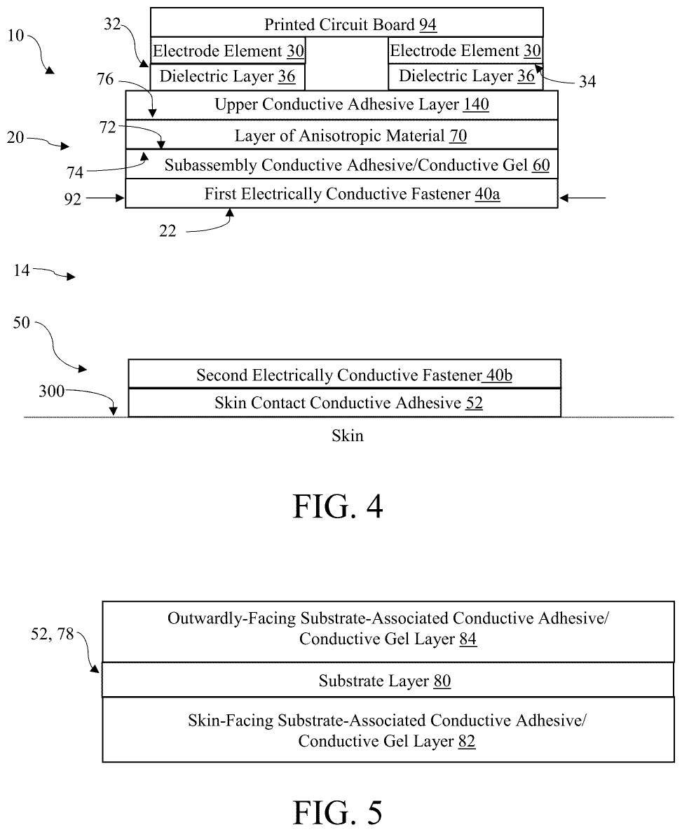

In various aspects, the apparatus 10 can comprise at least one sheet of anisotropic conductive material 70. For example, referring to FIGS. 2 and 3, in some aspects, the skin contact subassembly 50 can comprise a sheet of anisotropic conductive material 70. Referring to FIGS. 3 and 4, the electrode subassembly 20 can comprise a sheet of anisotropic conductive material 70. In exemplary aspects, and as illustrated in FIG. 3, both the skin contact subassembly 50 and the electrode subassembly 20 can comprise a respective sheet of anisotropic conductive material 70. The sheet of anisotropic conductive material 70 can have a skin-facing side 72, a skin-facing surface 74, and an opposing outwardly facing surface 76.

In some aspects, the sheet of anisotropic conductive material 70 can be or can comprise a synthetic graphite. In additional aspects, the sheet of anisotropic conductive material 70 can be or can comprise a layer (e.g., a sheet) of pyrolytic graphite, graphitized polymer film, or graphite foil made from compressed high purity exfoliated mineral graphite.

The sheet of anisotropic conductive material 70 can have a first thermal conductivity in a direction that is perpendicular to the skin facing surface. In some optional aspects, thermal conductivity of the sheet in directions that are parallel to the skin facing surface can be more than two times higher than the first thermal conductivity. The sheet of anisotropic conductive material 70 can further have a first resistance in a direction that is perpendicular to the skin facing surface. In some optional aspects, resistance of the sheet in directions that are parallel to the skin facing surface is less than half of the first resistance.

In aspects in which the skin contact subassembly 50 comprises a sheet of anisotropic conductive material 70, the sheet of anisotropic conductive material 70 can be disposed in contact with the electrically conductive skin contact layer 52. In such embodiments, the at least one electrode element 30 can be in electrical contact with the outwardly facing surface 78 of the sheet of anisotropic conductive material 70 when the electrode subassembly 20 is in contact with the skin contact subassembly 50.

Referring to FIGS. 3-4, in aspects in which the electrode subassembly 20 comprises a sheet of anisotropic conductive material 70, the electrode subassembly conductive adhesive or conductive gel layer 60 can be between the first electrically conductive fastener 40a and the sheet of anisotropic conductive material 70 (e.g., optionally, disposed against the skin-facing surface 74 of the sheet of anisotropic conductive material 70). In further aspects, the electrode subassembly 20 can comprise an upper conductive adhesive layer 140 positioned between the at least one electrode element 30 and the sheet of anisotropic conductive material 70 (e.g., optionally, disposed against the outwardly-facing surface 76 of the sheet of anisotropic conductive material 70).

In various aspects, the electrically conductive skin contact layer 52 can comprise an electrically conductive skin contact adhesive or an electrically conductive skin contact gel. For example, the electrically conductive skin contact layer can comprise a conductive adhesive composite. The conductive adhesive composite can comprise conductive particles dispersed therethrough. The conductive particles can comprise carbon. For example, the conductive particles can comprise one or more of: graphite powder, carbon flakes, carbon fibers, carbon granules, carbon nanotubes, carbon nanowires, carbon black powder, or carbon microcoils. In other aspects, the conductive particles can comprise metal.

Referring to FIGS. 2 and 3, in some aspects, the skin contact subassembly can comprise a three-layer unit comprising the electrically conductive skin contact layer 52, a sheet of anisotropic conductive material 70, and a second conductive adhesive or conductive gel layer 78. The skin facing surface 74 of the sheet of anisotropic conductive material can be in contact with the electrically conductive skin contact layer 52. The outwardly facing surface 76 of the sheet of anisotropic conductive material 70 can be in contact with the second conductive adhesive or conductive gel layer 78.

In some aspects, one or both of the electrically conductive skin contact layer 52 and the second conductive adhesive or conductive gel layer 78 can each consist of a single layer. In other aspects, and with reference to FIG. 5, one or both of the electrically conductive skin contact layer 52 and the second conductive adhesive or conductive gel layer 78 can comprise a substrate layer 80, a skin-facing substrate-associated conductive adhesive or conductive gel layer 82, and an outwardly-facing substrate-associated conductive adhesive or conductive gel layer 84.

In some aspects, the substrate layer 80 can have a continuous, uninterrupted structure, and the substrate layer can be electrically conductive. In other aspects, the substrate layer 80 can have an at least partially open structure that is configured to permit contact of adhesive layers 82, 84 from either side of the substrate layer. In exemplary aspects, the substrate 80 can comprise a mesh or a scrim.

In some aspects, one or more of the second conductive adhesive or conductive gel 78, the skin-facing substrate-associated conductive adhesive or conductive gel layer 82, or the outwardly-facing substrate-associated conductive adhesive or conductive gel layer 84 can be a conductive adhesive composite, as described elsewhere herein.

The electrode subassembly 20 can have an operative conductive area. For example, as shown in FIG. 1, in aspects in which the electrode subassembly 20 comprises a sheet of anisotropic conductive material 70 (e.g., FIGS. 3 and 4), the operative conductive area 90 can be defined by the outer perimeter of the anisotropic material. In aspects in which the electrode subassembly 20 does not comprise a sheet of anisotropic conductive material 70 (e.g., FIG. 2), the operative conductive area 90′ can be defined by an outer perimeter that surrounds every electrode of the at least one electrode (e.g., an areal footprint of the at least one electrode element). In some aspects, the skin contact subassembly 50 can overlie an entirety of the operative conductive area 90 of the electrode subassembly 20. In further aspects, the skin contact subassembly 50 can extend past the operative conductive area 90. For example, the skin contact subassembly 50 can extend past the operative conductive area 90 by at least 1 mm, or at least 2 mm, or at least 5 mm, or at least 10 mm or more. In further aspects, the skin contact subassembly 50 can fit within the operative conductive area 90.

In some aspects, the first electrically conductive fastener 40a (e.g., the hook or loop material or reversible reclosable fastener of the electrode subassembly 20) overlies an entirety of the areal footprint (constrained within the operative conductive area 90′) of the at least one electrode element. In further aspects, in which the electrode subassembly 20 comprises a sheet of anisotropic conductive material 70, the first electrically conductive fastener 40a can overlie an entirety of the operative conductive area 90 of the electrode subassembly 20.

The first electrically conductive fastener 40a can define a second areal footprint 92 (FIG. 4). In some aspects, the skin contact subassembly 50 can overlie an entirety of the second areal footprint 92. For example, the skin contact subassembly 50 can extend past the second areal footprint 92 defined by the first electrically conductive fastener 40a by at least 1 mm, or at least 2 mm, or at least 5 mm, or at least 10 mm or more. In this way, the first electrically conductive fastener 40a can be prevented from direct contact with the skin of the user. In other aspects, the skin contact subassembly 50 can fit within the operative conductive area 90.

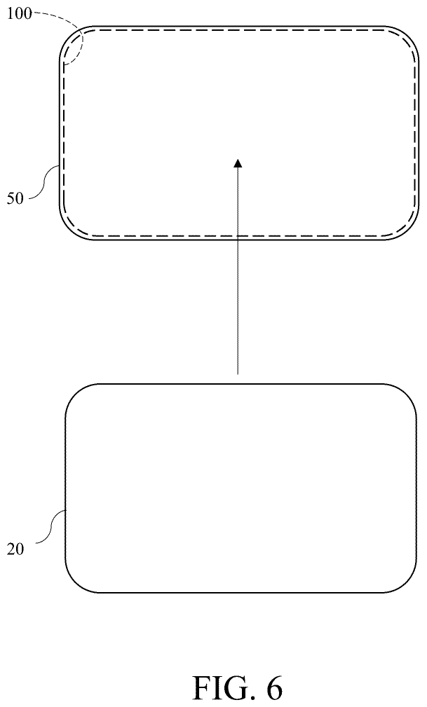

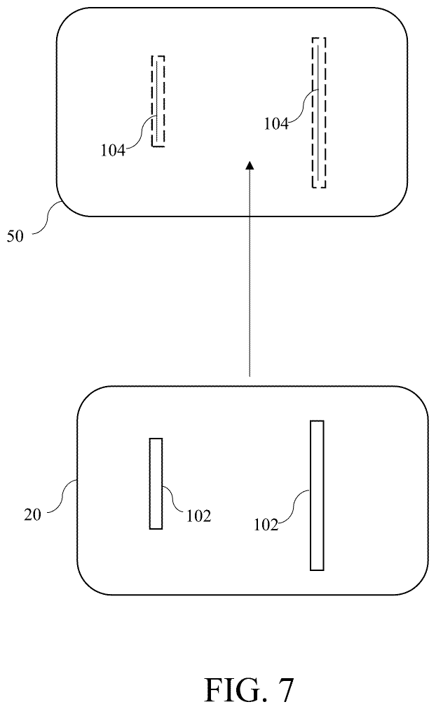

Referring to FIGS. 6-8, the skin contact subassembly 50 can comprise at least one alignment feature 100 that provides an indication of a desired position of the electrode subassembly 20 relative to the skin contact subassembly, in which the electrode subassembly is positioned within the outer periphery of the skin contact subassembly (and, conversely, the skin contact subassembly 50 has an areal footprint that overlies an entirety of the operative conductive area 90, shown in FIG. 1). For example, referring to FIG. 6, the alignment feature 100 on the skin contact subassembly 50 can comprise an outline of at least a portion of a perimeter of the electrode subassembly 20. In other aspects, and with reference to FIG. 7, the electrode subassembly 20 can define at least one opening 102 therethrough. The alignment feature 100 can be an opening 102 and the skin contact subassembly 50 can comprise a corresponding marking 104 on the skin contact subassembly 50 that is viewable through each opening 102 of the at least one opening through the electrode subassembly 20 when the electrode subassembly is in the desired position relative to the skin contact subassembly.

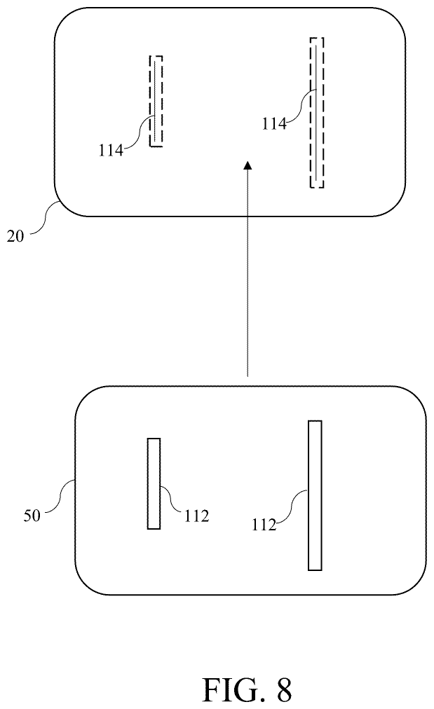

In some aspects, and with reference to FIG. 8, the skin contact subassembly 50 can define at least one opening 112 therethrough. The alignment feature 100 can comprise the at least one opening. The electrode subassembly 20 can comprise a corresponding marking 114 that is viewable through each opening 112 of the at least one opening through the skin contact subassembly 50 when the skin contact subassembly is in the desired position relative to the electrode subassembly 20.

In exemplary aspects, the electrode subassembly conductive adhesive or conductive gel 60 can have a thickness from about 40 μm to about 2000 μm. In exemplary aspects, the skin contact subassembly 50, omitting the second electrically conductive fastener 40b, can have a thickness from about 100 μm to about 400 μm, such as, for example, 200-250 μm.

In some optional aspects, the apparatus 10 can comprise a plurality of electrode elements 30. In these aspects, the electrode elements 30 can be wired together (e.g., using wires, traces on a printed circuit board 94 that can optionally be a flex circuit, etc.). In other aspects, the apparatus 10 can have only a single electrode element.

In various aspects, and as further described herein, the conductive adhesives disclosed herein can comprise a conductive adhesive composite, a hydrogel, or other suitable conductive material. For example, in some aspects, one or more of the conductive adhesives 52, 60, 78, 140 can comprise hydrogel.

Further, as discussed above, it is contemplated that one or more (optionally, all) of the conductive adhesive layers 52, 60, 78, 140 disclosed herein can comprise conductive adhesive composites (described further below) rather than hydrogel (such that hydrogel is not present in those adhesive layers). In exemplary aspects, the conductive adhesive composite can comprise a dielectric material and conductive particles dispersed within the dielectric material. In some embodiments, at least a portion of the conductive particles can define a conductive pathway through a thickness of the conductive adhesive composite. In some embodiments, it is contemplated that the conductive particles can be aligned in response to application of an electric field such that the conductive particles undergo electrophoresis. In some aspects, the dielectric material of the electrode assemblies is a polymeric adhesive. Optionally, in these aspects, the polymeric adhesive can be an acrylic adhesive. In some aspects, the conductive particles can comprise carbon. Optionally, in these aspects, the conductive particles can comprise graphite powder. Additionally, or alternatively, the conductive particles can comprise carbon flakes. Additionally, or alternatively, the conductive particles can comprise carbon granules. Additionally, or alternatively, the conductive particles can comprise carbon nanotubes. Additionally, or alternatively, the conductive particles can comprise carbon black powder. Additionally, or alternatively, the conductive particles can comprise carbon microcoils. In further aspects, the conductive adhesive composite further comprises a polar material (e.g., a polar salt). The polar salt can be a quaternary ammonium salt, such as a tetra alkyl ammonium salt. Exemplary conductive adhesive composites, as well as methods for making such conductive adhesive composites, are disclosed in U.S. Pat. No. 8,673,184 and U.S. Pat. No. 9,947,432, which are incorporated herein by reference for all purposes. In exemplary aspects, the conductive adhesive composite can be a dry carbon/salt adhesive, such as the OMNI-WAVE™ adhesive compositions manufactured and sold by FLEXCON® (Spencer, MA, USA). In further exemplary aspects, it is contemplated that the conductive adhesive composite can comprise a layer of an electrically conductive adhesive, such as for example, from use (by removal of the transfer film layer) of Electrically Conductive Adhesive Transfer Tape 9712 or Electrically Conductive Adhesive Transfer Tape 9713 (both manufactured by 3M).

A method of using the apparatus 10 can comprise removing the skin contact subassembly 50 from the skin-facing surface of the electrode subassembly 20. The method can further comprise positioning a new skin contact subassembly against the skin-facing surface 22 of the electrode subassembly 20 so that the new skin contact subassembly is removably coupled to the electrode subassembly by mechanical coupling.

In some aspects, the mechanical coupling can be operative using corresponding sides of electrically conductive fastener. For example, the mechanical coupling can be operative using corresponding sides of electrically conductive hook and loop material on each side of the electrode subassembly and the skin contact subassembly, or using corresponding sides of an electrically conductive reversible reclosable fastener on each side of the electrode subassembly and the skin contact subassembly

The method can further comprise cleaning the skin-facing surface of the electrode subassembly prior to positioning the new skin contact subassembly against the skin-facing surface of the electrode subassembly.

In aspects in which the electrode subassembly comprises at least one alignment feature that provides an indication of a desired position for the electrode subassembly relative to the skin contact subassembly (or vice-versa), the method can comprise using the at least one alignment feature to orient the electrode subassembly relative to the skin contact subassembly, or vice-versa.

A kit can comprise an electrode subassembly 20 comprising at least one electrode element 30 having a skin-facing side 32 and a skin-facing surface 34. The electrode subassembly 20 can have a skin-facing surface 22. The kit can further comprise a plurality of skin contact subassemblies 50, each comprising an electrically conductive skin contact layer 52. The electrically conductive skin contact layer 52 can be configured to contact skin 300 of a subject. Each skin contact subassembly 50 of the plurality of skin contact subassemblies can be configured to be removably coupled to the electrode subassembly 20 by mechanical coupling so that the electrically conductive skin contact layer 52 is electrically coupled to the at least one electrode 30 when the electrode subassembly is coupled to the skin contact subassembly.

A system can comprise an electrode subassembly 20 comprising at least one electrode element 30 having a skin-facing side 32 and a skin-facing surface 34. The electrode subassembly 20 can have a skin-facing surface 22. The system can further comprise a skin contact subassembly 50 comprising an electrically conductive skin contact layer 52. The electrically conductive skin contact layer 52 can be configured to contact skin 300 of a subject. The skin contact subassembly 50 can be configured to be removably coupled to the electrode subassembly 20 by mechanical coupling so that the electrically conductive skin contact layer 52 is electrically coupled to the at least one electrode 30 when the electrode subassembly is coupled to the skin contact subassembly.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus comprising:
an electrode subassembly comprising:
   at least one electrode element having a skin-facing side and a skin-facing surface, wherein the electrode subassembly has a skin-facing surface; and
a skin contact subassembly removably coupled to the skin-facing surface of the electrode subassembly by a mechanical coupling, wherein the skin contact subassembly comprises an electrically conductive skin contact layer configured to contact skin of a subject, and
wherein, when the skin contact subassembly is disposed against the skin-facing surface of the electrode subassembly, the electrically conductive skin contact layer is electrically coupled to the at least one electrode element.

Aspect 2: The apparatus of aspect 1, wherein the mechanical coupling is or comprises an electrically conductive fastener.

Aspect 3: The apparatus of aspect 1, wherein the electrode subassembly comprises one of hook or loop material, wherein the skin contact subassembly comprises the other of hook or loop material, wherein the one of the hook or loop material of the electrode subassembly and the other of the hook or loop material of the skin contact subassembly are engaged to define the mechanical coupling.

Aspect 4: The apparatus of aspect 3, wherein the one of the hook or loop material of the electrode subassembly comprises metal or comprises a coating comprising metal.

Aspect 5: The apparatus of aspect 3, wherein the one of the hook or loop material of the electrode subassembly comprises composite material having conductive particles dispersed therethrough or comprises a coating having conductive particles dispersed therethrough.

Aspect 6: The apparatus of aspect 5, wherein the conductive particles comprise carbon.

Aspect 7: The apparatus of aspect 6, wherein the conductive particles comprise one or more of: graphite powder, carbon flakes, carbon fibers, carbon granules, carbon nanotubes, carbon nanowires, carbon black powder, or carbon microcoils.

Aspect 8: The apparatus of aspect 5, wherein the conductive particles comprise metal.

Aspect 9: The apparatus of any one of aspects 3-8, wherein the other of the hook or loop material of the skin contact subassembly comprises metal or comprises a coating comprising metal.

Aspect 10: The apparatus of any one of aspects 3-8, wherein the other of the hook or loop material of the skin contact subassembly comprises composite material having conductive particles dispersed therethrough or comprises a coating having conductive particles dispersed therethrough.

Aspect 11: The apparatus of aspect 10, wherein the conductive particles comprise carbon.

Aspect 12: The apparatus of aspect 11, wherein the conductive particles comprise one or more of: graphite powder, carbon flakes, carbon fibers, carbon granules, carbon nanotubes, carbon nanowires, carbon black powder, or carbon microcoils.

Aspect 13: The apparatus of aspect 10, wherein the conductive particles comprise metal.

Aspect 14: The apparatus of any one aspects 3-13, wherein the one of hook or loop material of the electrode subassembly has an outwardly facing surface that faces the at least one electrode, and wherein the electrode subassembly further comprises an electrode subassembly conductive adhesive or conductive gel layer disposed against the outwardly facing surface of the one of hook or loop material.

Aspect 15: The apparatus of aspect 1, wherein the electrode subassembly comprises a first layer of a reversible reclosable fastener, wherein the skin contact subassembly comprises a corresponding second layer of a reversible reclosable fastener, wherein the first layer of the reversible reclosable fastener of the electrode subassembly and the corresponding second layer of the reversible reclosable fastener of the skin contact subassembly are engaged to define the mechanical coupling.

Aspect 16: The apparatus of aspect 15, wherein the first layer of the reversible reclosable fastener of the electrode subassembly and/or the corresponding second layer of the reversible reclosable fastener of the skin contact subassembly comprises metal or comprises a coating comprising metal.

Aspect 17: The apparatus of aspect 15, wherein the first layer of the reversible reclosable fastener of the electrode subassembly and/or the corresponding second layer of the reversible reclosable fastener of the skin contact subassembly comprises composite material having conductive particles dispersed therethrough or comprises a coating having conductive particles dispersed therethrough.

Aspect 18: The apparatus of aspect 17, wherein the conductive particles comprise carbon.

Aspect 19: The apparatus of aspect 18, wherein the conductive particles comprise one or more of: graphite powder, carbon flakes, carbon fibers, carbon granules, carbon nanotubes, carbon nanowires, carbon black powder, or carbon microcoils.

Aspect 20: The apparatus of aspect 17, wherein the conductive particles comprise metal.

Aspect 21: The apparatus of any one aspects 15-20, wherein the first layer of the reversible reclosable fastener of the electrode subassembly has an outwardly facing surface that faces the at least one electrode, and wherein the electrode subassembly further comprises an electrode subassembly conductive adhesive or conductive gel layer disposed against the outwardly facing surface of the first layer of the reversible reclosable fastener.

Aspect 22: The apparatus of any one of aspects 1-21, wherein the electrode subassembly further comprises a dielectric layer on the skin-facing side of the at least one electrode element.

Aspect 23: The apparatus of any one of aspects 1-21, wherein the electrode subassembly further comprises a dielectric layer on the skin-facing surface of the at least one electrode element.

Aspect 24: The apparatus of aspect 22 or aspect 23, wherein the dielectric layer comprises a ceramic or dielectric polymer.

Aspect 25: The apparatus of aspect 24, wherein the dielectric layer comprises a dielectric polymer, and wherein the dielectric polymer has a dielectric constant greater than 10.

Aspect 26: The apparatus of any one of the preceding aspects, wherein the apparatus does not comprise adhesive between the skin contact subassembly and the electrode subassembly.

Aspect 27: The apparatus of any one of the preceding aspects, wherein the electrode subassembly and the skin contact subassembly are removably coupled by a physical, non-chemical adhesion.

Aspect 28: The apparatus of any one of the preceding aspects, wherein the skin contact subassembly further comprises a sheet of anisotropic conductive material.

Aspect 29: The apparatus of aspect 28, wherein the sheet of anisotropic conductive material has a skin-facing side with a skin-facing surface and an opposing outwardly facing surface, wherein the sheet of anisotropic conductive material is disposed in contact with the electrically conductive skin contact layer, and wherein the at least one electrode element is in electrical contact with the outwardly facing surface of the sheet of anisotropic conductive material when the electrode subassembly is in contact with the skin contact subassembly.

Aspect 30: The apparatus of aspect 28 or aspect 29, wherein the sheet of anisotropic conductive material is or comprises a synthetic graphite.

Aspect 31: The apparatus of aspect 28 or aspect 29, wherein the sheet of anisotropic conductive material is or comprises a layer of pyrolytic graphite, graphitized polymer film, or graphite foil made from compressed high purity exfoliated mineral graphite.

Aspect 32: The apparatus of aspect 28 or aspect 29, wherein the sheet of anisotropic conductive material has a first thermal conductivity in a direction that is perpendicular to the skin facing surface, and wherein thermal conductivity of the sheet in directions that are parallel to the skin facing surface is more than two times higher than the first thermal conductivity.

Aspect 33: The apparatus of aspect 28 or aspect 29, wherein the sheet of anisotropic conductive material has a first resistance in a direction that is perpendicular to the skin facing surface, and wherein resistance of the sheet in directions that are parallel to the skin facing surface is less than half of the first resistance.

Aspect 34: The apparatus of any one of the preceding aspects, wherein the electrically conductive skin contact layer comprises an electrically conductive skin contact adhesive or an electrically conductive skin contact gel.

Aspect 35: The apparatus of any one of the preceding aspects, wherein the electrically conductive skin contact layer comprises a conductive adhesive composite.

Aspect 36: The apparatus of aspect 35, wherein the conductive adhesive composite comprises conductive particles dispersed therethrough.

Aspect 37: The apparatus of aspect 36, wherein the conductive particles comprise carbon.

Aspect 38: The apparatus of aspect 37 wherein the conductive particles comprise one or more of: graphite powder, carbon flakes, carbon fibers, carbon granules, carbon nanotubes, carbon nanowires, carbon black powder, or carbon microcoils.

Aspect 39: The apparatus of aspect 36, wherein the conductive particles comprise metal.

Aspect 40: The apparatus of any one of the preceding aspects, wherein the skin contact subassembly comprises a three-layer unit comprising the electrically conductive skin contact layer, a sheet of anisotropic conductive material, and a second conductive adhesive or conductive gel layer; wherein the sheet of anisotropic conductive material has a skin facing side with a skin facing surface and an opposing outwardly facing surface; and wherein the skin facing surface of the sheet of anisotropic conductive material is in contact with the electrically conductive skin contact layer, and the outwardly facing surface of the sheet of anisotropic conductive material is in contact with the second conductive adhesive or conductive gel layer.

Aspect 41: The apparatus of aspect 40, wherein one or both of the electrically conductive skin contact layer and the second conductive adhesive or conductive gel layer comprise:
a substrate layer;
a skin-facing substrate-associated conductive adhesive or conductive gel layer; and
an outwardly-facing substrate-associated conductive adhesive or conductive gel layer.

Aspect 42: The apparatus of aspect 41, wherein the substrate layer has a continuous, uninterrupted structure, and wherein the substrate layer is electrically conductive.

Aspect 43: The apparatus of aspect 41, wherein the substrate layer has an at least partially open structure that is configured to permit contact of adhesive layers from either side of the substrate layer.

Aspect 44: The apparatus of aspect 43, wherein the substrate comprises a mesh or a scrim.

Aspect 45: The apparatus of aspect 41, wherein one or more of the second conductive adhesive or conductive gel, the skin-facing substrate-associated conductive adhesive or conductive gel layer or the outwardly-facing substrate-associated conductive adhesive or conductive gel layer is a conductive adhesive composite.

Aspect 46: The apparatus of aspect 45, wherein the conductive adhesive composite comprises conductive particles dispersed therethrough.

Aspect 47: The apparatus of aspect 46, wherein the conductive particles comprise carbon.

Aspect 48: The apparatus of aspect 47, wherein the conductive particles comprise one or more of: graphite powder, carbon flakes, carbon fibers, carbon granules, carbon nanotubes, carbon nanowires, carbon black powder, or carbon microcoils.

Aspect 49: The apparatus of aspect 46, wherein the conductive particles comprise metal.

Aspect 50: The apparatus of any one of the preceding aspects, wherein the electrode subassembly has an operative conductive area, wherein the skin contact subassembly has an areal footprint that overlies an entirety of the operative conductive area.

Aspect 51: The apparatus of any one of the preceding aspects, wherein the electrode subassembly has an operative conductive area, the skin contact subassembly has an areal footprint that overlies an entirety of the operative conductive area, and the skin contact subassembly comprises at least one alignment feature that provides an indication of a desired position of the electrode subassembly relative to the skin contact subassembly.

Aspect 52: The apparatus of aspect 51, wherein the alignment feature comprises an outline of at least a portion of a perimeter of the electrode subassembly.

Aspect 53: The apparatus of aspect 51, wherein the electrode subassembly defines at least one opening therethrough, wherein the alignment feature comprises a corresponding marking on the skin contact subassembly that is viewable through each opening of the at least one opening through the electrode subassembly when the electrode subassembly is in the desired position relative to the skin contact subassembly.

Aspect 54: The apparatus of aspect 51, wherein the skin contact subassembly defines at least one opening therethrough, wherein the alignment feature comprises the at least one opening, wherein the electrode subassembly comprises a corresponding marking that is viewable through each opening of the at least one opening through the skin contact subassembly when the skin contact subassembly is in the desired position relative to the electrode subassembly.

Aspect 55: The apparatus of any one of aspects 3-54, wherein the at least one electrode element defines an areal footprint, wherein the one of hook or loop material of the electrode subassembly overlies an entirety of the areal footprint of the at least one electrode element; or wherein the first layer of the reversible reclosable fastener of the electrode subassembly overlies an entirety of the areal footprint of the at least one electrode element.

Aspect 56: The apparatus of any one of aspects 3-55, wherein the one of hook or loop material of the electrode subassembly defines a second areal footprint and the skin contact subassembly overlies an entirety of the second areal footprint; or wherein the first layer of the reversible reclosable fastener of the electrode subassembly defines a second areal footprint and the skin contact subassembly overlies an entirety of the second areal footprint.

Aspect 57: The apparatus of any one of aspects 35-56, wherein the electrically conductive skin contact layer comprises an electrically conductive skin contact adhesive or an electrically conductive skin contact gel.

Aspect 58: The apparatus of aspect 1, wherein the electrode subassembly comprises at least one projection, protrusion, slot, hook, fastener, snap, or interlocking feature that is configured to engage a complementary projection, protrusion, slot, hook, fastener, snap, or interlocking feature to define the mechanical coupling.

Aspect 59: A method of using an apparatus as in any one of the preceding aspects, the method comprising:
removing the skin contact subassembly from the skin-facing surface of the electrode subassembly.

Aspect 60: The method of aspect 59, further comprising positioning a new skin contact subassembly against the skin-facing surface of the electrode subassembly so that the new skin contact subassembly is removably coupled to the electrode subassembly by mechanical coupling.

Aspect 61: The method of aspect 60, wherein the mechanical coupling is operative using corresponding sides of hook and loop material on each side of the electrode subassembly and the skin contact subassembly, or using corresponding sides of a reversible reclosable fastener on each side of the electrode subassembly and the skin contact subassembly.

Aspect 62: The method of aspect 60, further comprising cleaning the skin-facing surface of the electrode subassembly prior to positioning the new skin contact subassembly against the skin-facing surface of the electrode subassembly.

Aspect 63: The method of aspect 60, wherein the skin contact subassembly comprises at least one alignment feature that provides an indication of a desired position of the electrode subassembly relative to the skin contact subassembly, the method comprising using the at least one alignment feature to orient the electrode subassembly relative to the skin contact subassembly, or wherein the electrode subassembly comprises at least one alignment feature that provides an indication of a desired position of the skin contact subassembly relative to the electrode subassembly, the method comprising using the at least one alignment feature to orient the skin contact subassembly relative to the electrode subassembly.

Aspect 64: A kit comprising:
an electrode subassembly comprising at least one electrode element having a skin-facing side and a skin-facing surface, wherein the electrode subassembly has a skin-facing surface; and
a plurality of skin contact subassemblies each comprising an electrically conductive skin contact layer, and wherein the electrically conductive skin contact layer is configured to contact skin of a subject,
wherein each skin contact subassembly of the plurality of skin contact subassemblies is configured to be removably coupled to the electrode subassembly by mechanical coupling so that the electrically conductive skin contact layer is electrically coupled to the at least one electrode element.

Aspect 65: The kit of aspect 64, wherein the mechanical coupling is operative using an electrically conductive fastener.

Aspect 66: The kit of aspect 64 or aspect 65, wherein the mechanical coupling is operative using corresponding sides of hook and loop material on each of the electrode subassembly and the skin contact subassembly, or using corresponding sides of a reversible reclosable fastener on each of the electrode subassembly and the skin contact subassembly.

Aspect 67: The kit of any one of aspects 64-66, wherein the skin contact subassemblies each comprise a three-layer unit comprising a skin contact conductive adhesive or conductive gel, a sheet of anisotropic conductive material, and a second conductive adhesive or conductive gel layer; wherein the sheet of anisotropic conductive material has a skin facing side with a skin facing surface and an opposing outwardly facing surface; and wherein the skin facing surface of the sheet of anisotropic conductive material is in contact with the skin contact conductive adhesive or conductive gel, and the outwardly facing surface of the sheet of anisotropic conductive material is in contact with the second conductive adhesive or conductive gel layer.

Aspect 68: The kit of aspect 67, wherein the sheet of anisotropic conductive material is or comprises a synthetic graphite.

Aspect 69: The kit of aspect 67, wherein the sheet of anisotropic conductive material is or comprises a layer of pyrolytic graphite, graphitized polymer film, or graphite foil made from compressed high purity exfoliated mineral graphite.

Aspect 70: A system comprising:
an electrode subassembly comprising at least one electrode element having a skin-facing side and a skin-facing surface, wherein the electrode subassembly has a skin-facing surface; and
a skin contact subassembly comprising an electrically conductive skin contact layer, and wherein the electrically conductive skin contact layer is configured to contact skin of a subject,
wherein the skin contact subassembly is configured to be removably coupled to the electrode subassembly by mechanical coupling so that the electrically conductive skin contact layer is electrically coupled to the at least one electrode element.

Aspect 71: The system of aspect 70, wherein the mechanical coupling is operative using an electrically conductive fastener.

Aspect 72: The system of aspect 70 or aspect 71, wherein the mechanical coupling is operative using corresponding sides of hook and loop material on each of the electrode subassembly and the skin contact subassembly, or using corresponding sides of a reversible reclosable fastener on each of the electrode subassembly and the skin contact subassembly.

Aspect 73: The system of any one of aspects 70-72, wherein the skin contact subassembly comprises a three-layer unit comprising a skin contact conductive adhesive or conductive gel, a sheet of anisotropic conductive material, and a second conductive adhesive or conductive gel layer; wherein the sheet of anisotropic conductive material has a skin facing side with a skin facing surface and an opposing outwardly facing surface; and wherein the skin facing surface of the sheet of anisotropic conductive material is in contact with the skin contact conductive adhesive or conductive gel, and the outwardly facing surface of the sheet of anisotropic conductive material is in contact with the second conductive adhesive or conductive gel layer.

Aspect 74: The system of aspect 73, wherein the sheet of anisotropic conductive material is or comprises a synthetic graphite.

Aspect 75: The system of aspect 73, wherein the sheet of anisotropic conductive material is or comprises a layer of pyrolytic graphite, graphitized polymer film, or graphite foil made from compressed high purity exfoliated mineral graphite.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus comprising:
an electrode subassembly comprising:
at least one electrode element having a skin-facing side and a skin-facing surface, wherein the electrode subassembly has a skin-facing surface; and
a skin contact subassembly removably coupled to the skin-facing surface of the electrode subassembly by a mechanical coupling, wherein the skin contact subassembly comprises an electrically conductive skin contact layer configured to contact skin of a subject, and
wherein, when the skin contact subassembly is disposed against the skin-facing surface of the electrode subassembly, the electrically conductive skin contact layer is electrically coupled to the at least one electrode element.

2. The apparatus of claim 1, wherein the mechanical coupling is or comprises an electrically conductive fastener.

3. The apparatus of claim 1, wherein the electrode subassembly comprises one of hook or loop material, wherein the skin contact subassembly comprises the other of hook or loop material, wherein the one of the hook or loop material of the electrode subassembly and the other of the hook or loop material of the skin contact subassembly are engaged to define the mechanical coupling.

4. The apparatus of claim 3, wherein each of the one of the hook or loop material of the electrode subassembly and the other of the hook or loop material of the skin contact subassembly comprises metal or comprises a coating comprising metal or comprises composite material having conductive particles dispersed therethrough or comprises a coating having conductive particles dispersed therethrough.

5. The apparatus of claim 3, wherein the one of hook or loop material of the electrode subassembly has an outwardly facing surface that faces the at least one electrode, and wherein the electrode subassembly further comprises an electrode subassembly conductive adhesive or conductive gel layer disposed against the outwardly facing surface of the one of hook or loop material.

6. The apparatus of claim 1, wherein the electrode subassembly comprises a first layer of a reversible reclosable fastener, wherein the skin contact subassembly comprises a corresponding second layer of a reversible reclosable fastener, wherein the first layer of the reversible reclosable fastener of the electrode subassembly and the corresponding second layer of the reversible reclosable fastener of the skin contact subassembly are engaged to define the mechanical coupling.

7. The apparatus of claim 6, wherein the first layer of the reversible reclosable fastener of the electrode subassembly and/or the corresponding second layer of the reversible reclosable fastener of the skin contact subassembly comprises metal or comprises a coating comprising metal or comprises composite material having conductive particles dispersed therethrough or comprises a coating having conductive particles dispersed therethrough.

8. The apparatus of claim 6, wherein the first layer of the reversible reclosable fastener of the electrode subassembly has an outwardly facing surface that faces the at least one electrode, and wherein the electrode subassembly further comprises an electrode subassembly conductive adhesive or conductive gel layer disposed against the outwardly facing surface of the first layer of the reversible reclosable fastener.

9. The apparatus of claim 1, wherein the electrode subassembly further comprises a dielectric layer on the skin-facing side of the at least one electrode element.

10. The apparatus of claim 1, wherein either the skin contact subassembly or the electrode subassembly, or both, further comprises a sheet of anisotropic conductive material.

11. The apparatus of claim 1, wherein the electrically conductive skin contact layer is a first conductive adhesive or conductive gel layer, wherein the skin contact subassembly comprises a three-layer unit comprising the first conductive adhesive or conductive gel layer, a sheet of anisotropic conductive material, and a second conductive adhesive or conductive gel layer, wherein the sheet of anisotropic conductive material has a skin-facing side with a skin-facing surface and an opposing outwardly facing surface, wherein the skin-facing side of the sheet of anisotropic conductive material is disposed in contact with the first conductive adhesive or conductive gel layer, and wherein the at least one electrode element is in electrical contact with the outwardly facing surface of the sheet of anisotropic conductive material when the electrode subassembly is in contact with the skin contact subassembly.

12. The apparatus of claim 11, wherein the sheet of anisotropic conductive material is or comprises a synthetic graphite.

13. The apparatus of claim 1, wherein the electrode subassembly comprises at least one projection, protrusion, slot, hook, fastener, snap, or interlocking feature that is configured to engage a complementary projection, protrusion, slot, hook, fastener, snap, or interlocking feature to define the mechanical coupling.

14. A method comprising:
removing a skin contact subassembly from a skin-facing surface of an electrode subassembly by separating a mechanical coupling between the skin contact subassembly and the electrode subassembly,
wherein the electrode subassembly comprises at least one electrode element having a skin-facing side and a skin-facing surface, wherein the electrode subassembly has a skin-facing surface,
wherein the skin contact subassembly comprises an electrically conductive skin contact layer configured to contact skin of a subject, and
wherein, prior to separating the mechanical coupling, the skin contact subassembly is disposed against the skin-facing surface of the electrode subassembly and the electrically conductive skin contact layer is electrically coupled to the at least one electrode element, and
positioning a new skin contact subassembly against the skin-facing surface of the electrode subassembly so that the new skin contact subassembly is removably coupled to the electrode subassembly by mechanical coupling, wherein the new skin contact subassembly comprises a new electrically conductive skin contact layer configured to contact skin of a subject, and wherein, when the new skin contact subassembly is removably coupled to the electrode subassembly, the new electrically conductive skin contact layer is electrically coupled to the at least one electrode element.

15. The method of claim 14, wherein the mechanical coupling is or comprises an electrically conductive fastener and is operative using corresponding sides of hook and loop material on each side of the electrode subassembly and the skin contact subassembly, or using corresponding sides of a reversible reclosable fastener on each side of the electrode subassembly and the skin contact subassembly.

16. A kit comprising:
an electrode subassembly comprising at least one electrode element having a skin-facing side and a skin-facing surface, wherein the electrode subassembly has a skin-facing surface; and
a plurality of skin contact subassemblies each comprising an electrically conductive skin contact layer, and wherein the electrically conductive skin contact layer is configured to contact skin of a subject, wherein each skin contact subassembly of the plurality of skin contact subassemblies is configured to be removably coupled to the electrode subassembly by mechanical coupling so that the electrically conductive skin contact layer is electrically coupled to the at least one electrode element.

17. The kit of claim 16, wherein the mechanical coupling is operative using an electrically conductive fastener.

18. The kit of claim 16, wherein the mechanical coupling is operative using corresponding sides of hook and loop material on each of the electrode subassembly and the skin contact subassembly, or using corresponding sides of a reversible reclosable fastener on each of the electrode subassembly and the skin contact subassembly.

19. The kit of claim 16, wherein the skin contact subassemblies each comprise a three-layer unit comprising a skin contact conductive adhesive or conductive gel, a sheet of anisotropic conductive material, and a second conductive adhesive or conductive gel layer; wherein the sheet of anisotropic conductive material has a skin facing side with a skin facing surface and an opposing outwardly facing surface; and wherein the skin facing surface of the sheet of anisotropic conductive material is in contact with the skin contact conductive adhesive or conductive gel, and the outwardly facing surface of the sheet of anisotropic conductive material is in contact with the second conductive adhesive or conductive gel layer.

20. The kit of claim 19, wherein the sheet of anisotropic conductive material is or comprises a synthetic graphite.
